(19) United States
(12) Patent Application Publication
(10) Pub. No.: US 2023/0346489 A1
(43) Pub. Date: Nov. 2, 2023

(54) POWER MANAGEMENT ARCHITECTURE FOR SURGICAL ROBOTIC SYSTEMS

(71) Applicant: Covidien LP, Mansfield, MA (US)

(72) Inventor: Edward J. Naclerio, Madison, CT (US)

(21) Appl. No.: 17/791,332

(22) PCT Filed: Jan. 25, 2021
(86) PCT No.: PCT/US2021/014873

§ 371 Date: Jul. 7, 2022

Related U.S. Application Data
(60) Provisional application No. 62/970,776, filed on Feb. 6, 2020.

(51) Int. Cl. A61B 34/30; A61B 50/13; A61B 90/50; B25J 5/00; B25J 19/00

(52) U.S. Cl. CPC A61B 34/30; A61B 50/13; A61B 90/50; B25J 5/007; B25J 19/0025

(57) ABSTRACT

A surgical robotic system includes at least one movable cart including a robotic arm having a surgical instrument. The surgical robotic system also includes a control tower including a power supply system coupled to the at least one movable cart via a cable. The power supply system includes: a power supply configured to output a voltage signal to power the at least one movable cart and at least one status signal; a cable state detection circuit configured to detect a connection signal indicative of a connection status of the cable; and a controller coupled to the cable state detection circuit and the power supply, the controller configured to control the power supply based on the connection status of the cable and the at least one status signal.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm. Such robotic systems are powered by complex electrical power supply systems with multiple electrical supply rails and backup units. Thus, there is a need for a streamlined power management for surgical robotic systems to control complex electrical power supplies.

### SUMMARY

The present disclosure provides a surgical robotic system including a plurality of components, namely, a control tower, a console, and one or more surgical robotic arms, each of which is disposed on a movable cart and includes a surgical instrument. The control tower includes a power supply system which distributes power to each of the movable carts and the robotic arms attached thereto. The power supply system includes a plurality of power supplies, each of which powers an individual movable cart. The power supply system includes a controller configured to control the individual power supply based on a connection status of the movable cart and a status of the individual power supply.

According to one embodiment of the present disclosure, a surgical robotic system is disclosed. The surgical robotic system includes at least one movable cart including a robotic arm having a surgical instrument. The surgical robotic system also includes a control tower including a power supply system coupled to the at least one movable cart via a cable. The power supply system includes: a power supply configured to output a voltage signal to power at least one movable cart and at least one status signal; a cable state detection circuit configured to detect a connection signal indicative of a connection status of the cable; and a controller coupled to the cable state detection circuit and the power supply, the controller configured to control the power supply based on the connection status of the cable and the at least one status signal.

According to one aspect of the above embodiment, the power supply includes a power supply connector and a communication connector, each of which is coupled to the controller. The power supply system also includes a power supply isolator coupled to the power supply connector and a communication isolator coupled to the communication connector.

According to another aspect of the above embodiment, the power supply system further includes an isolation barrier galvanically isolating the power supply from the controller.

According to another embodiment of the present disclosure, a surgical robotic system is disclosed. The surgical robotic system includes a plurality of movable carts each including a robotic arm having a surgical instrument and a control tower including a power supply system, which includes a plurality of power supplies each of which is coupled via a cable to one movable cart of the plurality of movable carts and is configured to output a voltage signal to power the one movable cart and at least one status signal; a plurality of cable state detection circuits each of which is configured to detect a connection signal indicative of a connection status of the cable; a plurality of controllers coupled to one cable state detection circuit of the plurality of the cable state detection circuits and one power supply of the plurality of power supplies, the controller configured to control the one power supply based on the connection status of the cable and at least one status signal; and a plurality of isolation barriers galvanically isolating each of the power supplies from each other and from each of the controllers.

According to one aspect of the above embodiment, each of the power supplies includes a power supply connector and a communication connector, each of which is coupled to the corresponding controller. Each of the isolation barriers includes a power supply isolator coupled to power supply connector and a communication isolator coupled to the communication connector.

According to one aspect of any of the above embodiments, the cable state detection circuit includes a debouncer.

According to another aspect of any of the above embodiments, the controller is further configured to terminate the voltage signal of the power supply in response to termination of the connection signal. The controller is further configured to terminate the voltage signal of the power supply in response to the at least one status signal being outside a predetermined parameter.

According to a further embodiment of the present disclosure, a method for controlling a surgical robotic system is disclosed. The method includes outputting a voltage signal from a power supply to power a movable cart including a robotic arm having a surgical instrument and transmitting a connection signal indicative of a connection status of a cable connecting a movable cart to the power supply. The method also includes transmitting at least one status signal from the power supply; and controlling, at a controller, the power supply based on the connection status of the cable and the at least one status signal.

According to one aspect of the above embodiment, the method further includes coupling the power supply to the controller through a power supply connector and a communication connector. The method also includes galvanically isolating the power supply from the controller through an isolation barrier. The method further includes coupling a power supply isolator to the power supply connector and a communication isolator to the communication connector.

According to another aspect of the above embodiment, the method further includes debouncing the connection signal through a debouncer.

According to yet another aspect of the above embodiment, the method further includes terminating the voltage signal of the power supply in response to termination of the connection signal.

According to a further aspect of the above embodiment, the method further includes terminating the voltage signal of the power supply in response to the at least one status signal being outside a predetermined parameter.

### BRIEF DESCRIPTION OF THE DRAWINGS

Embodiments of the present disclosure are described herein with reference to the accompanying drawings, wherein:

FIG. 1 is a schematic illustration of a surgical robotic system including a control tower, a console, and one or more surgical robotic arms according to the present disclosure;

FIG. 2 is a perspective view of a surgical robotic arm of the surgical robotic system of FIG. 1 according to the present disclosure;

FIG. 3 is a perspective view of a setup arm with the surgical robotic arm of the surgical robotic system of FIG. 1 according to the present disclosure;

FIG. 4 is a schematic diagram of a computer architecture of the surgical robotic system of FIG. 1 according to the present disclosure;

FIG. 5 is a schematic diagram of a power supply system according to the present disclosure; and

FIG. 6 is a schematic diagram of a tower power supply chassis of the power supply system of FIG. 5 according to one embodiment of the present disclosure;

FIG. 7 is a schematic diagram of a tower power supply chassis of the power supply system of FIG. 5 according to another embodiment of the present disclosure;

FIG. 8 is a schematic diagram of a control circuit of the power supply system of FIG. 5 for controlling AC input according to one embodiment of the present disclosure;

FIG. 9 is a schematic diagram of a control circuit of the power supply system of FIG. 5 for controlling AC input according to another embodiment of the present disclosure; and

FIG. 10 is a schematic diagram of a control circuit of the power supply system of FIG. 5 for controlling AC input according to yet another embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

As will be described in detail below, the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

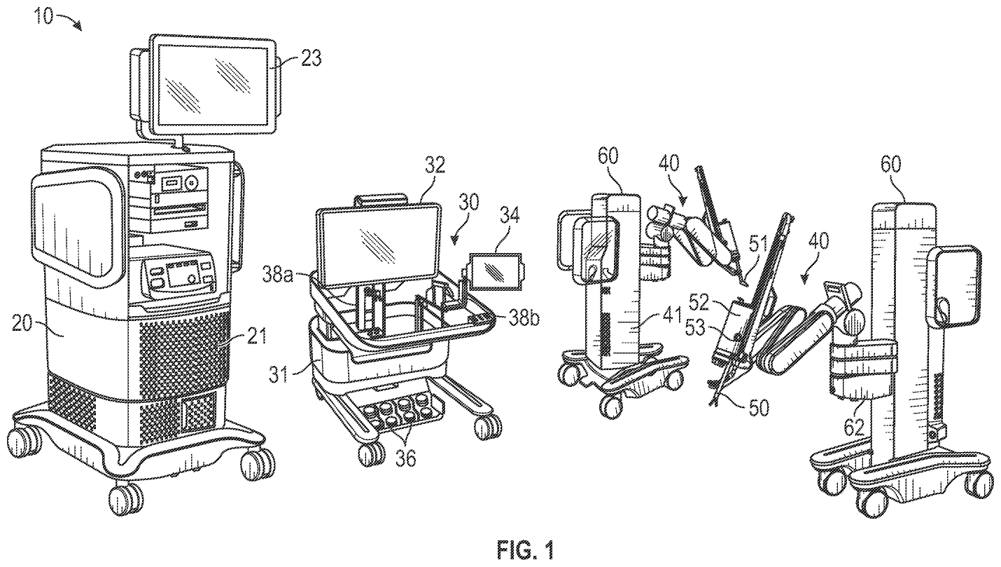

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope configured to provide a video feed for the user. In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

Each of the robotic arms 40 may include a camera 51 configured to capture video of the surgical site. The camera 51 may be a stereoscopic camera and may be disposed along with the surgical instrument 50 on the robotic arm 40. The surgical console 30 includes a first display 32, which displays a video feed of the surgical site provided by camera 51 of the surgical instrument 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b which are used by a user to remotely control robotic arms 40.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

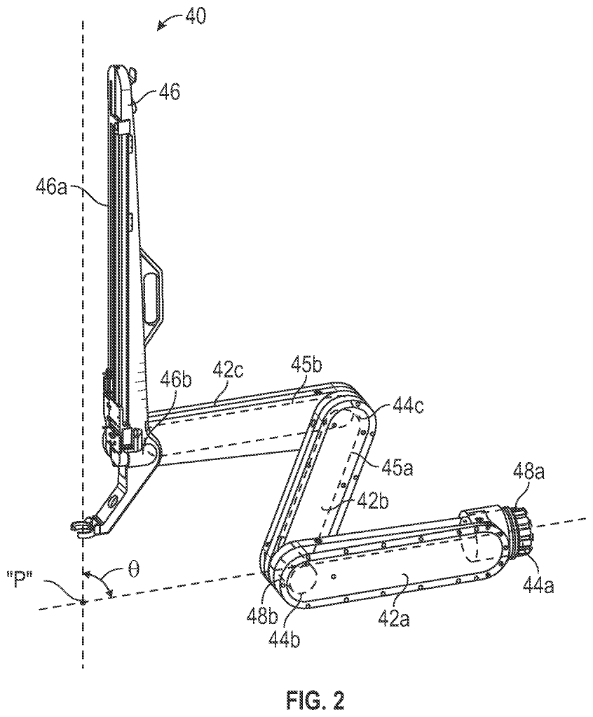

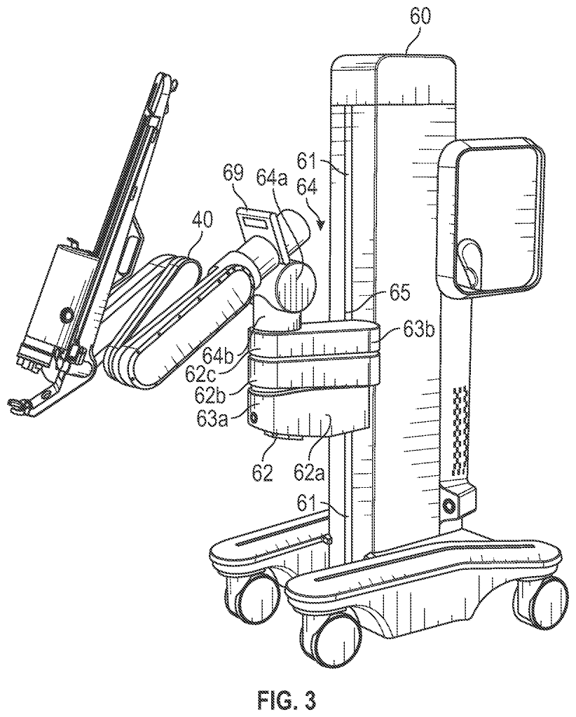

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The robotic arm 40 also includes a plurality of manual override buttons 53 disposed on instrument drive unit 52 and the setup arm 62, which may be used in a manual mode. The user may press one of the buttons 53 to move the component associated with the button 53.

With reference to FIG. 2, the robotic arm 40 also includes a holder 46 defining a second longitudinal axis and configured to receive the instrument drive unit 52 (FIG. 1) of the surgical instrument 50, which is configured to couple to an actuation mechanism of the surgical instrument 50. Instrument drive unit 52 transfers actuation forces from its actuators to the surgical instrument 50 to actuate components (e.g., end effectors) of the surgical instrument 50. The holder 46 includes a sliding mechanism 46a, which is configured to move the instrument drive unit 52 along the second longitudinal axis defined by the holder 46. The holder 46 also includes a joint 46b, which rotates the holder 46 relative to the link 42c.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

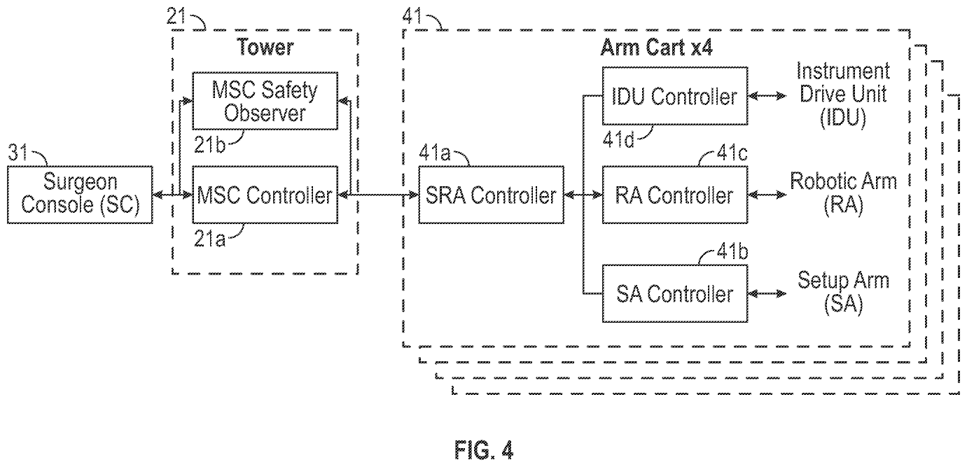

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

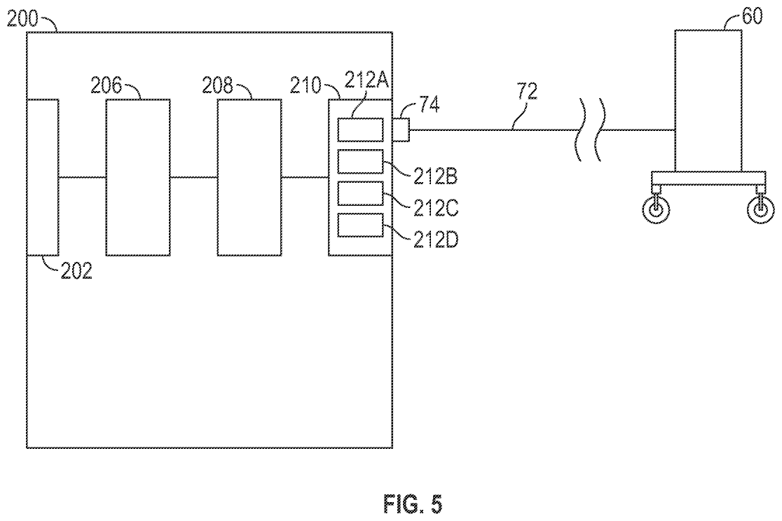

With reference to FIG. 5, the robotic system 10 includes a power supply system 200 housed in the control tower 20. Each of movable carts 60 is electrically coupled to the power supply system 200 via a cable 72 having a connector 74. The power supply system 200 includes a power ingress module 202 coupled to an electrical main supplying alternating current and an isolation transformer 206. The power supply system 200 also includes one or more uninterruptible power supply (“UPS”) 208 coupled to the isolation transformer 206. The UPS 208 provide backup electrical power and is coupled to a tower power supply chassis (“TPSC”) 210. In embodiments, the TPSC 210 includes a plurality of power supplies 212a-d configured to provide a regulated DC output to each of the movable carts 60. Power supplies 212a-d may be AC/DC converters. Thus, the TPSC 210 includes a plurality of power supplies 212a-d, one for each of the movable cart 60 such that each of the power supplies 212a-d supplies power to a single movable cart 60.

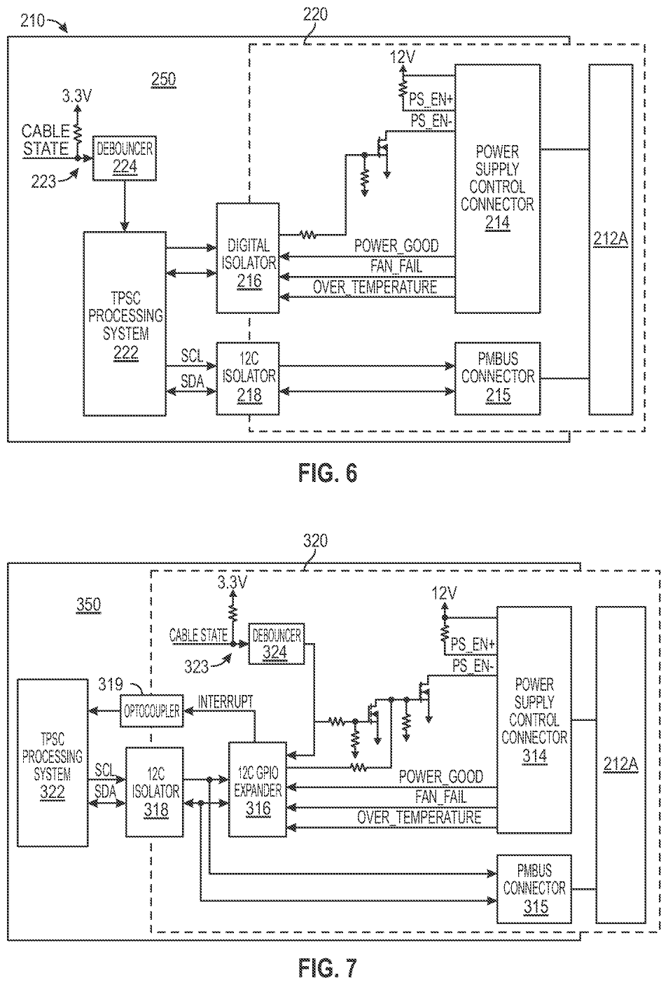

With reference to FIG. 6, the TPSC 210 may be disposed on a printed circuit board assembly (“PCBA”) 250 having a plurality of power control components to deliver controlled DC output to the movable carts 60. Each of the power supplies 212a-d includes a power supply control connector 214 and a communication connector 215. For simplicity, only the power supply 212a is shown. The power supply control connector 214 transmits control and status signals to enable the main power DC output. Status signals may include power status of the power supply 212a, fan status of the fans cooling the power supply 212a, and over-temperature status of the temperature of power supply 212a. The communication connector 215 may be part of any suitable communication bus with the controller 21, such as the PMBus interface, SMBus interface, I²C interface, and the like, allowing the controller 21 to monitor the output voltage, current, and temperature of the power supply 212a. The power supply 212a may include a plurality of outputs, such as a main power DC output for powering the movable cart 60. The output may be from about 24 volts to about 48 volts. The power supply 212a may also include a peripheral output, which may be about 12 volts, and 300 milliamps, to power various circuit components of the TPSC 210. The peripheral output is used to power to an isolation barrier 215, which includes a digital isolator 216 and a communication bus isolator 218, which galvanically isolate the power supply 212a. The isolation barrier 215 ensures the output from each of the power supplies 212a is floating relative to each other as well as relative to a protective earth connection. This allows the isolation barrier 215 to limit the occurrence of single fault conditions from impacting more than one movable cart 60.

With continued reference to FIG. 6, an isolation barrier 220 is shown as a dashed border. Peripheral output of the power supply 212a provides power for the isolation barrier 215, which is supplied through the power supply control connector 214. The peripheral output may be at a first voltage level e.g., about 12 VDC, and may be regulated to a lower voltage, e.g., about 3.3 VDC, to power the digital isolator 216 and the communication bus isolator 218. The TPSC 210 includes a TPSC controller 222, which communicates with the digital isolator 216 and the communication bus isolator 218. The digital isolator 216 enables the TPSC controller 222 to control the output of the power supply 212a-d. In particular, the TPSC controller 222 outputs a “power supply output enable” (PS_EN) signal based on the status signals from the power supply 212a. The digital isolator 216 enables the TPSC controller 222 to read the output voltage, current, and temperature of the power supply 212a. The TPSC controller 222 also includes a cable state detection circuit 223, which monitors a cable state signal from the movable cart 60 to determine if the movable cart 60 is connected to the TPSC 210 via the connector 74 of the cable 72 (FIG. 5). The movable cart 60 periodically outputs the cable state signal while the movable cart 60 is connected to the TPSC 210. The cable state detection circuit 223 includes a debouncer 224, which generates a clean digital signal to limit the noise due to intermittent connection of the connector 74. Thus, the TPSC controller 222 controls the output of the power supply 212a based on the cable state signal, the status signals, and output of the power supply 212a. In particular, if the cable state signal is interrupted and/or one of the status signals or the output signals is outside a predetermined parameter, the TPSC controller 222 terminates output of the power supply 212a.

The TPSC 210 includes an individual isolation barrier 220 for each of the movable carts 60. Each of the isolation barrier 220 are independent from one another, and independent of the rest of the PCBA 250, if any single fault occurs, up to one Power supply 212a-d is impacted. If any failure of the PCBA 250 occurs, such as power supply failure on the PCBA 250, loss of power, or complete software cessation, the output state of the power supply 212a that existed prior to the failure is lost. Further, disconnecting the cable 72 connecting the movable cart 50 to the TPSC 210 will shut off the output of the power supply 212a.

FIG. 7, shows another embodiment of a PCBA 350, which is substantially similar to the PCBA 250 and thus description of similar components, e.g., isolation barrier 320, communication bus isolator 318, TPSC controller 322, cable state detection circuit 323, debouncer 324, their functionality, and signals transmitted therethrough is omitted. In the PCBA 350, a TPSC controller 322 is completely isolated, with the cable state signal being included in the isolation barrier 320.

The isolation barrier 320 includes an input/output expander 316, which is coupled to the cable state detection circuit 323 having the debouncer 324 for receiving the cable state signal from the movable cart 60. The debouncer 324 prevents intermittent connection or noise of the cable state signal from shutting off the output of the power supply 212a. The input/output expander 316 is also coupled to the power supply control connector 314 and the communication connector 315 in parallel. The input/output expander 316 is coupled to the TPSC controller 322 through a communication bus isolator 318 and an optocoupler 319, which provide for galvanic separation. The input/output expander 316 enables the TPSC controller 322 to read the status signals, the output voltage, current, and temperature of the power supply 212a, which are used by the TPSC controller 322 to control the output of the power supply 212a. In particular, the TPSC controller 322 controls the output of the power supply 212a by outputting on PS_EN signal.

Peripheral output of the power supply 212a provides power for the isolation barrier 320, which is supplied through the power supply control connector 314a. The peripheral output may be at a first voltage level e.g., about 12 VDC, and may be regulated to a lower voltage, e.g., about 3.3 VDC, to power the input/output expander 316 and the communication bus isolator 318.

When the cable state signal indicates the movable cart 60 is attached to the TPSC 210, the TPSC controller 322 activates the power supply 212a by controlling the output state of a signal driven by the input/output expander 316. Therefore, the state of the PS_EN signal depends on both the cable state signal and the software running on the TPSC controller 322. If the cable between the movable cart 60 and the TSPC 210 is disconnected, the output of the power supply 212a is turned off automatically. Even if the TPSC controller 322 ceases operation, the software programmed value for determining the state of the PS_EN signal remains unchanged and the power supply 212a continues to supply power to the attached movable cart 60.

In embodiments, the input/output expander 316 may include an interrupt output that is activated if any of the digital inputs supplied to the input/output expander 316 change state. This configuration may be used provide TPSC controller 322 with an alternative to polling for monitoring the status signals of the power supply 212a. In further embodiments, the input/output expander 316 may be replaced by a microcontroller. The functionality of the input/output expander 316 may be replicated by the software running on the microcontroller, which may also be used to offer additional functionality.

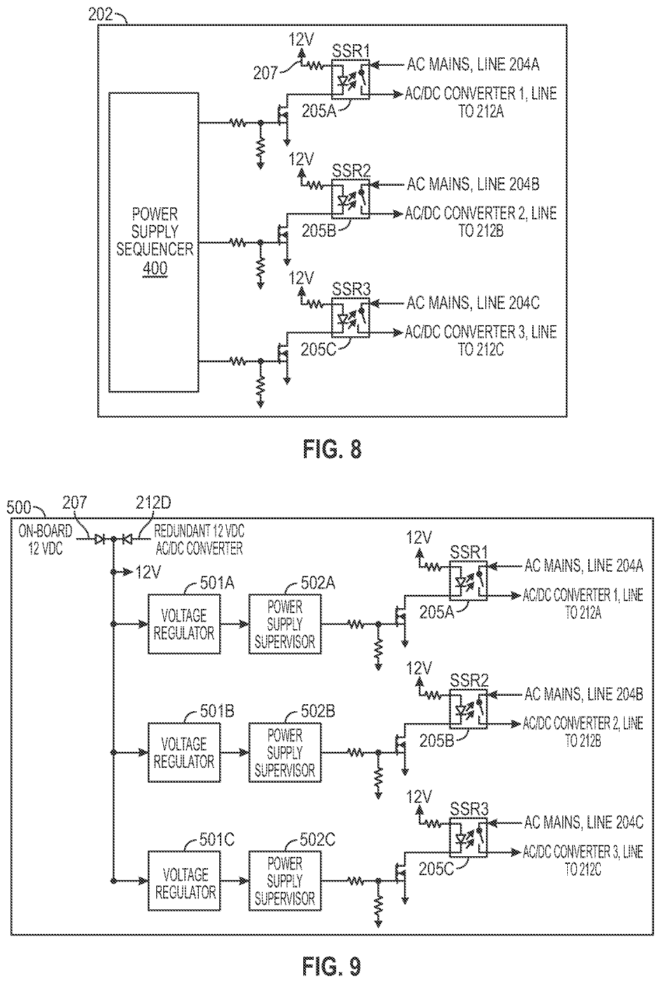

With reference to FIG. 8, the power ingress module 202 includes a power sequencer 400, which is coupled to three AC line inputs 204a, 204b, 204c. The power sequencer 400 staggers AC line inputs 204a, 204b, 204c to three power supplies 212a-c to limit the in-rush current to the TPSC 210. The power supply 212d is connected directly to the AC input to the TPSC 210 and is not connected to the power sequencer 400 and is not shown in FIG. 8. The power ingress module 202 also includes a plurality of solid state relays (SSRs) 205a, 205b, 205c, each of which is coupled to the three AC line inputs 204a, 204b, 204c, respectively. The SSRs 205a-c may be activated by a 12 VDC signal supplied by an on-board power supply 207 of the TPSC 210. The power sequencer 400 drives signals to SSRs 205a-c that connect AC line inputs 204a-c to the AC inputs of the power supplies 212a-c. The timing of the signals is such the period of high current that occurs when powering up each power supply 212a-c is non-overlapping.

With reference to FIG. 9, another control circuit 500 for controlling the SSRs 205a-c is shown. Power for the control circuit 500 is redundantly supplied by the on-board power supply 207, as well as the power supply 212d. The redundant supply mitigates the loss of power that controls the SSRs 205a-c. In addition, three power supply supervisors 502a, 502b, 502c, each with progressively longer hardware configured timing, control the SSRs 205a-c, respectively, that turn on the Power supplies 212a-c. The control circuit 500 also includes three voltage regulators 501a, 501b, 501c, each of which is coupled to the power supply supervisors 502a, 502b, 502c. The voltage regulators 501a, 501b, 501c regulate power supplied to the power supply supervisors 502a, 502b, 502c, which operate at a lower voltage than the 12 VDC supplied to the control circuit 500. Since each power supply supervisor 502a-c is independent from the others, there is no potential single point failure.

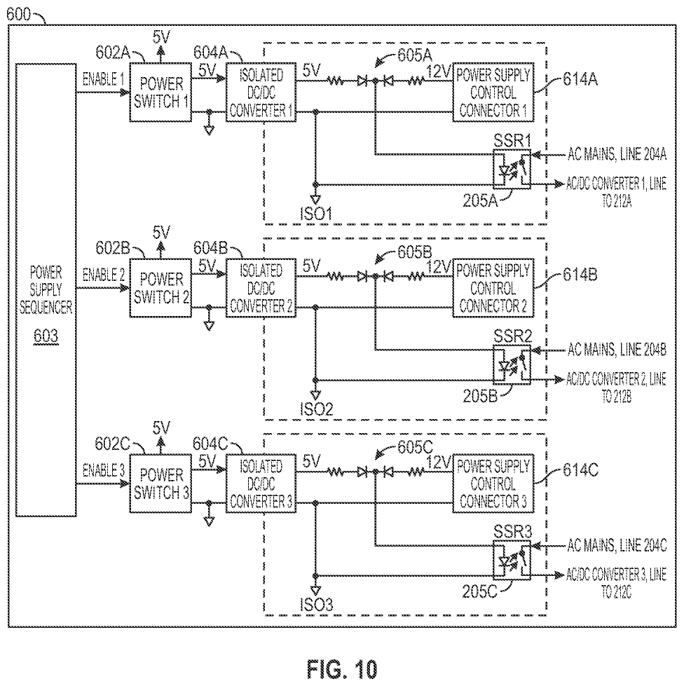

With reference to FIG. 10, another control circuit 600 for controlling the SSRs 205a-c is shown. The control circuit 600 includes a power sequencer 603, which sequentially turns on each of power switches 602a-c, which in turn supply power to isolated DC/DC converters 604a-c. The output on the isolated side of each of the DC/DC converters 604a-c then activates an off-board SSRs 205a-c. As each SSR 205a-c is turned on, its corresponding power supply 212a-c powers up and activates its output through a corresponding power supply control connector 614a-c, which are similar to the power supply control connector 214 described above. The outputs of the DC/DC converters 604a-c and the corresponding power supplies 212a-c are supplied to a diode-OR circuit 605a-c thereby isolating the voltage sources from each other. Thereafter, the output from each of the DC/DC converters 604a-c keeps corresponding SSR 205a-c turned on, there is no potential single point failure. Each power supply 212a-c remains on even after any loss of power to any of the isolated DC/DC converters 604a-c.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
at least one movable cart including a robotic arm having a surgical instrument; and
a control tower including a power supply system coupled to the at least one movable cart via a cable, the power supply system including:
a power supply configured to output a voltage signal to power the at least one movable cart and at least one status signal;
a cable state detection circuit configured to detect a connection signal indicative of a connection status of the cable; and
a controller coupled to the cable state detection circuit and the power supply, the controller configured to control the power supply based on the connection status of the cable and the at least one status signal.

2. The surgical robotic system according to claim 1, wherein the power supply includes a power supply connector and a communication connector, each of which is coupled to the controller.

3. The surgical robotic system according to claim 2, wherein the power supply system includes a power supply isolator coupled to the power supply connector and a communication isolator coupled to the communication connector.

4. The surgical robotic system according to claim 1, wherein the power supply system further includes an isolation barrier galvanically isolating the power supply from the controller.

5. The surgical robotic system according to claim 1, wherein the cable state detection circuit includes a debouncer.

6. The surgical robotic system according to claim 1, wherein the controller is further configured to terminate the voltage signal of the power supply in response to termination of the connection signal.

7. The surgical robotic system according to claim 1, wherein the controller is further configured to terminate the voltage signal of the power supply in response to the at least one status signal being outside a predetermined parameter.

8. A surgical robotic system comprising:
a plurality of movable carts each including a robotic arm having a surgical instrument; and
a control tower including a power supply system including:
a plurality of power supplies each of which is coupled via a cable to one movable cart of the plurality of movable carts and is configured to output a voltage signal to power the one movable cart and at least one status signal;
a plurality of cable state detection circuits each of which is configured to detect a connection signal indicative of a connection status of the cable;
a plurality of controllers coupled to one cable state detection circuit of the plurality of the cable state detection circuits and one power supply of the plurality of power supplies, the controller configured to control the one power supply based on the connection status of the cable and the at least one status signal; and
a plurality of isolation barriers galvanically isolating each of the power supplies from each other and from each of the controllers.

9. The surgical robotic system according to claim 8, wherein each of the power supplies includes a power supply connector and a communication connector, each of which is coupled to the corresponding controller.

10. The surgical robotic system according to claim 9, wherein each of the isolation barriers includes a power supply isolator coupled to power supply connector and a communication isolator coupled to the communication connector.

11. The surgical robotic system according to claim 8, wherein each of the cable state detection circuits includes a debouncer.

12. The surgical robotic system according to claim 8, wherein each of the controllers is configured to terminate the voltage signal of the corresponding power supply in response to termination of the connection signal.

13. The surgical robotic system according to claim 8, wherein each of the controllers is configured to terminate the voltage signal of the corresponding power supply in response to the at least one status signal being outside a predetermined parameter.

14. A method for controlling a surgical robotic system, the method comprising:
outputting a voltage signal from a power supply to power a movable cart including a robotic arm having a surgical instrument;
transmitting a connection signal indicative of a connection status of a cable connecting a movable cart to the power supply;
transmitting at least one status signal from the power supply; and
controlling, at a controller, the power supply based on the connection status of the cable and the at least one status signal.

15. The method according to claim 14, further comprising coupling the power supply to the controller through a power supply connector and a communication connector.

16. The method according to claim 15, further comprising galvanically isolating the power supply from the controller through an isolation barrier.

17. The method according to claim 16, wherein galvanically isolating further includes coupling a power supply isolator to the power supply connector and a communication isolator to the communication connector.

18. The method according to claim 14, further comprising debouncing the connection signal through a debouncer.

19. The method according to claim 14, terminating the voltage signal of the power supply in response to termination of the connection signal.

20. The method according to claim 14, terminating the voltage signal of the power supply in response to the at least one status signal being outside a predetermined parameter.
